(19) United States
(12) Patent Application Publication
(10) Pub. No.: US 2002/0103365 A1
(43) Pub. Date: Aug. 1, 2002

(54) PROCESS FOR THE SYNTHESIS OF NUCLEIC ACIDS ON A SOLID SUPPORT AND COMPOUNDS WHICH ARE USEFUL IN PARTICULAR AS SOLID SUPPORTS IN THE SAID PROCESS

(76) Inventors: LUDMILLA BARANOVA, VILLEMONBLE (FR); FRANCOIS CHATELAIN, PARIS (FR); VIKTOR KUMAREV, VILLEMONBLE (FR)

Correspondence Address: FRANK C. EISENSCHENK, Ph. D., SALIWANCHIK, LLOYD & SALIWANCHIK, 2421 N.W. 41ST STREET, SUITE A-1, GAINESVILLE, FL, 32606-6669, US

(21) Appl. No.: 09/076,956

(22) Filed: May 13, 1998

Related U.S. Application Data
(62) Division of application No. 08/591,466, filed on Jan. 11, 1996, now abandoned.
Application No. 08/591,466 is a 371 of international application No. PCT/FR94/00842, filed on Jul. 7, 1994.

(30) Foreign Application Priority Data
Jul. 9, 1993 (FR) 93 08498

(51) Int. Cl. C07H 21/00; C07H 21/02; C07H 21/04

(52) U.S. Cl. 536/25.3; 536/25.33; 536/25.34

(57) ABSTRACT

The object of the present invention is a process for synthesizing solid phase nucleic acids, characterized in that a mineral or organic polymer, bound by a bivalent hydrocarbon radical to an epoxy or glycol-type group, is used as a solid support, said epoxy or glycol-type group comprising two adjacent saturated carbon atoms on which an OH and a nucleophilic group are substituted. The present invention also pertains to compounds containing an epoxy or glycol-type group as defined above, useful for example as a solid support in a process for solid support nucleic acid synthesis.

The present invention relates to a process for the synthesis of nucleic acids on a solid support. The present invention also relates to a solid support which is useful, in particular, in biotechnology and particularly in the process for the synthesis of nucleic acids according to the invention.

The present invention lastly relates to a process for the preparation of the said solid support.

The synthesis of nucleic acids on a solid support is used particularly in the automated synthess of DNA or RNA oligonucleotides.

In the present Application, the terms “nucleic acids” is understood to refer to deoxyribonucleic acids or ribonucleic acids or, more generally, polynucleotides or oligonucleotides in which the bases, internucleotide phosphate bonds or the ribose rings of the bases may be chemically modified in a known manner. They may in particular be oligonucleotides of α or β anomers, oligonucleotides of internucleotidic bonding of the phosphorothioate or methyl phosphonate type, or alternatively oligothionucleotides.

The first step of a process for the synthesis of nucleic acids on a solid support consists in attaching the first nucleoside of the desired sequence to a solid support, traditionally consisting of glass beads of controlled porosity (CPG) or, more generally, of a functionalized organic or inorganic polymer.

The techniques currently used involve the use of eight different reagents as solid supports, consisting of a functionalized organic or inorganic polymer bound to an A, T, C, G or U nucleoside, depending on whether the sequence to be prepared contains A, T, C, G or U as the first deoxyribo- or ribonucleotide. Moreover, manufacturers supply reactors in which one of these nucleosides has already been attached to the support. The appropriate reactor is thus selected depending on whether the sequence begins with A, T, C, G or U. Elongation of this first nucleoside then takes place in the 3′→5′ or 5′→3′ direction, by means of coupling reagents. One synthetic cycle, that is to say the coupling between two nucleotides, includes at least three steps: (1) deprotection of the 5′ or 3′ OH function of a first nucleotide, in particular detritylation, (2) activation of the said 5′ or 3′ OH function of this first nucleotide and condensation with the 3′ or 5′ end respectively of a second nucleotide, and, lastly, (3) oxidation of the phosphite group of the internucleotide bond obtained to phosphate.

The oligonucleotide is preferably synthesized in the 3′→5′ direction. In this case, the starting material is a 5′ OH-protected nucleoside attached to the support via the 3′ end of the deoxyribose or ribose ring. The nucleotides which are subsequently added are in the form of a 5′-protected derivative whose 3′ hydroxyl possesses a substituted phosphite or phosphate group.

Different methods are distinguished depending on the type of substitution on the phosphate: the phosphoramidite method, described in particular in EP 61,746 and U.S. Pat. No. 4,458,066, is nowadays one of the methods of choice since it makes it possible to achieve high coupling yields (greater than 98%). The 3′ hydroxyl thus possesses a phosphoramidite group (see FIG. 1). Besides the importance of these groups for the solubility of the nucleosides in the organic solvent, the phosphoramidite group renders the phosphorus atom more susceptible to attack by a primary hydroxyl function, such as that in the 5′ position of the detritylated growing nucleosides or chains. The deprotected 5′ hydroxyl function becomes sufficiently nucleophilic to react with the phosphoramidite group of the second nucleotide.

The solid phase syntheses of DNA and RNA have great similarities. The monomers and the supports are different but the instrumentation and the reagents are identical.

The oligonucleotides obtained at the end of the synthetic cycles must be detached from the support and the protective functions must be removed. Cleavage of the support, deprotection of the bases and removal of the group bonded to the phosphorus are carried out simultaneously in aqueous ammonia solution. In the case of RNA, ethanol makes it possible to solubilize the 2′-O-silyl-oligoribonucleotides and to minimize the desilylation, native RNA not being stable under basic conditions. The aqueous ammonia/ethanol solution containing the oligoribonucleotide which has passed into the liquid phase is then separated from the glass support and evaporated. Removal of the silyl groups takes place in the presence of tetrabutylammonium fluoride (TBAF) at room temperature for sixteen hours. The TBAF is then neutralized with TEAA (triethylammonium acetate).

Other methods also exist, in particular the so-called phosphotriester method, phosphodiester method, H-phosphonate method and, lastly, phosphite method.

A solid support which may be used for the automated synthesis of oligonucleotides must satisfy the following characteristics:

1) the solid support must react selectively with the functionalized 3′ end of the nucleotide in particular of the phosphoramidite, H-phosphonate, phosphotriester, phosphodiester or phosphite type or with any other monomer reagent according to the synthetic method used;

2) the support-oligonucleotide bond must be stable under the conditions of the synthesis, and

3) the support-oligonucleotide bond must be able to be hydrolyzed at the end of the synthesis under the conditions for the step of deprotection of the oligonucleotide, and

4) the covalent bond between the support and oligonucleotide must be such that, during the detachment, the released oligonucleotide is of native type, that is to say that the 3′ terminal hydroxyl function is free or does not bear any residue derived from the synthesis.

Many supports have already been described in the literature for the solid phase synthesis of oligonucleotides.

These supports may consist of organic polymers such as polystyrene (Nucleic A. Res. 1980, volume 8), polyacrylamide acryloylmorpholide, polydimethyl-acrylamide polymerized on kieselguhr (Nucleic Ac. Res. 9(7) 1691 (1980)).

Other supports described are of inorganic nature, in particular based on silica functionalized with a hydrocarbon radical bearing an NH₂ and/or COOH group (J. Am. Chem., 105, 661 (1983), or the support based on silica functionalized with a 3-aminopropyltriethoxysilane group whose use in phosphite and phosphoramidite synthesis for the preparation of oligonucleotides was described for the first time in European patent No. 0,035,719.

However, these supports have significant defects: they are not universal and can only be used in oligonucleotide synthesis after prior preparation of the corresponding nucleoside derivatives thereof, for example CPG-A, CPG-G, CPG-T, CPG-C or CPG-Da, CPG-dG, CPG-dU, CPG-Dc; the preparation of these derivatives also involves a prior preparation of the 3′-p-nitrophenyl-succinate-nucleoside which requires more time and considerable expense of reagent.

In order to fulfil the four conditions described above, and in particular the last one, the supports currently used are bound to the first ribonucleoside or deoxyribonucleoside of the sequence to be synthesized, as described above. In particular, there is no phosphate group between the 3′ (or 5′) end of the first nucleotide or nucleoside and the functionalized polymer. In order to start the synthesis, the operator must thus select from among supports corresponding in general to a formula as follows:

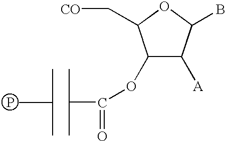

in which:

A is a hydrogen atom (deoxyribonucleoside) or an optionally protected hydroxyl group (ribonucleoside),

B is a purine or pyrimidine base whose exocyclic amide function is optionally protected. These protective agents, generally benzoyl or isobutyryl, also assist in the solubilization thereof in the organic solvents used in the course of the synthesis,

C is the usual temporary protecting group for the 5′ terminal function, in general of the trityl type such as dimethoxytrityl,

P is the solid support consisting of an organic or inorganic polymer connected directly to the 3′ end, optionally substituted with a divalent hydrocarbon radical connected via an ester bond in the 3′ position of the nucleoside.

One aim of the present invention is to provide a process for the solid phase synthesis of oligonucleotides, more particularly a process of automatic synthesis, in which a so-called “universal” support is used. The expression “universal support” refers here to a solid support which may be used irrespective of the first RNA or DNA nucleotide to be synthesized, and irrespective of the type of monomer reagent used during the synthesis, that is to say irrespective of the type of substitution on the phosphate group in the 3′ position or in the 5′ position depending on whether the synthesis is carried out in the 5′→3′ or 3′→5′ direction.

Another aim of the present invention is to be able to use this “universal support” in a process involving the same reaction conditions as in the automated solid phase syntheses.

In particular, one aim of the present invention is that the monomer reagent serving to attach the first nucleotide to the solid support should be a monomer reagent identical to the monomer reagent serving to attach the other nucleotides of the sequence during the synthesis, in particular as regards the 5′ protection and the 3′ protection.

Another aim is also that the solid support should be in accordance with the four characteristics mentioned above.

In particular, one difficulty in the aim that the present invention wishes to address resides in the fact that the first nucleotide which is introduced contains a 3′ or 5′ phosphate group which must, after cleavage between the support and the oligonucleotide under the usual conditions of deprotection in basic medium, be capable at the end of the synthesis of liberating an end 3′ or 5′ OH, depending on the case.

To make such a universal support was hitherto considered as inconceivable on account of the apparent incompatibility between the need to synthesize a 3′ OH oligonucleotide, for example, and the direct use, from the very first base, of a reagent identical to the usual monomer reagents bearing a phosphate group in the terminal 3′ position.

According to the present invention, we have succeeded in functionalizing the polymer of the solid support with a hydrocarbon radical containing a reactive group such that:

1) the group can be coupled to a protected 3′ or 5′ end of the monomer reagents, under the same conditions as those for which the 3′ or 5′ end of the terminal nucleotide in the chain already synthesized are coupled with the 5′ or 3′ end respectively of the next monomer reagent to be attached, and

2) the final cleavage of the covalent bond between the solid support and the oligonucleotide, via this group, takes place under the conditions of the final deprotection of the oligonucleotide, and

3) the hydroxyl function at the terminal 3′ or 5′ end can be free or, more generally, such that the terminal phosphate group of the first nucleotide remains on the support.

The solid phase “universality” of the supports according to the present invention is obtained by means of a functionalization of the inorganic or organic polymer with a hydrocarbon radical containing groups of the glycol type in which an OH group and a nucleophilic group are vicinally arranged, that is to say located on two adjacent carbons, at the end of the hydrocarbon radical, it being optionally possible for these two carbons to be substituted with inert groups.

The expression “inert group” refers here to a group which does not react under the conditions encountered during the various steps of the synthesis according to the invention of nucleic acids on a solid support.

The subject of the present invention is thus a process for the preparation of nucleic acids by synthesis on a solid support, characterized in that an inorganic or organic polymer is used as solid support, which polymer is connected via a divalent hydrocarbon radical to an epoxide group or a group of the glycol type, the latter group consisting of two adjacent saturated carbons on which an OH group and a nucleophilic group are respectively substituted.

The first nucleotide is advantageously attached to the solid support under the same conditions and with the same monomer reagent as for the condensation of the second nucleotide with the first nucleotide bonded to the support, which may be the conventional conditions and monomer reagents used during the synthesis of nucleic acids on a solid support, the said first nucleotide corresponding to the first nucleotide in the sequence of the said nucleic acid.

In one particular embodiment, the process of the invention comprises the following steps of:

1) condensation of the 5′ or 3′ OH group of the first nucleotide or of an oligonucleotide connected at its other 3′ or 5′ end to the said solid support, using a coupling agent, with the phosphate group optionally substituted in the 3′ or 5′ position respectively of a nucleotide monomer reagent protected in the 3′ and 5′ positions;

2) oxidation or sulfurization of the internucleotide bond of the phosphite type obtained in step 1) to a phosphate bond respectively.

3) deprotection of the 5′-O or 3′-O end of the product obtained in step 2);

4) repetition of steps 1) to 3) as many times as there are nucleotides to be added in order to synthesize the nucleic acid.

More precisely, the process may comprise the following steps of:

1) condensation, using a coupling agent, of the said OH group of the said group of glycol type of the solid support with a phosphate or phosphite group optionally substituted in the 3′ or 5′ position of a nucleotide monomer reagent protected in the 5′ O and 3-O positions;

2) oxidation or sulfurization of the covalent bond of the phosphite type between the solid support and the first nucleotide obtained in step 1);

3) deprotection of the 5′-O or 3′-O end of the product obtained in step 2);

4) condensation of the 5′ OH or 3′ OH group of the product obtained in step 3) with the phosphate, phosphorothioate or phosphite group optionally substituted in the 3′ or 5′ position of a nucleotide monomer reagent protected in the 5′-O or 3′ -O position respectively, using the said coupling agent, under the same conditions as in step 1);

5) oxidation or sulfurization of the internucleotide grouping of the phosphite phosphite [sic] type resulting from the above step into a grouping of the phosphate or phosphorothioate type respectively;

6) deprotection of the 5′-O or 3′-O end of the product obtained in step 5);

7) repetition of steps (4), (5) and (6) as many times as there are nucleotides to be added in order to obtain the nucleic acid to be prepared.

The above steps lead to an oligonucleotide connected to the solid support. In an appropriate manner, the process according to the invention includes a final step of detachment of the nucleic acid from the support and removal of the protecting groups from the bases and, where appropriate, from the 2′-O positions of the nucleic acid.

In the prior techniques in which the solid support is already connected to a first nucleoside corresponding to the first nucleotide of the sequence to be prepared, before starting the synthetic cycles, the said support generally contains a protection of the said nucleoside in the 5′ or 3′ position. In this case, the synthetic cycle begins with a step of deprotection in acid medium, generally a detritylation with TFA, DCA or TCA in dichloromethane.

According to the present invention, the process may also begin with a deprotection step and a support according to the invention containing an epoxide group may then be used as initial solid support.

The process according to the invention comprises in this case a prior step of opening of the said epoxide group of the said solid support, in an anhydrous acidic medium, under the usual conditions for the deprotection of the 5′ or 3′ OH groups in order to give the said group of the glycol type of the solid support.

Another subject of the present invention is compounds of the following formulae and their use as solid supports in a process for the synthesis of nucleic acids according to the invention:

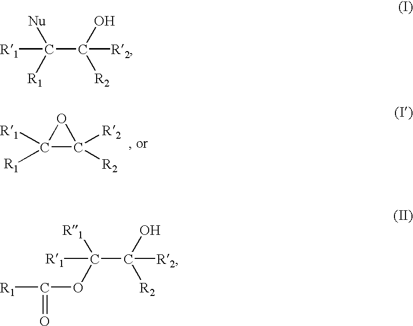

in which:

one of R₁, R′₁, R′′₁, R₂and R′₂represents an inorganic or organic polymer— [symbol] or a hydrocarbon radical substituted with an inorganic or organic polymer,and the others represent H or an inert group such as an alkyl group which is optionally substituted, in particular with one or more halogen(s),

Nu is a nucleophilic group such as NH₂, —O—Alk, —NHAlk, —N(Alk)₂, —NHAc, —OAc, —S—Ac, —S—Alk or Halogen; the groups Alk and Ac being C₁ to C₇, preferably C₁ to C₄alkyl and acyl groups respectively, which are optionally substituted, in particular with one or more halogen(s). Mention is made more particularly of the compounds for which Nu is —N(Alk)₂, —NHAc, —O—Ac, —SAc and a halogen.

In an appropriate embodiment, the said solid support takes up [sic ] one of the formulae:

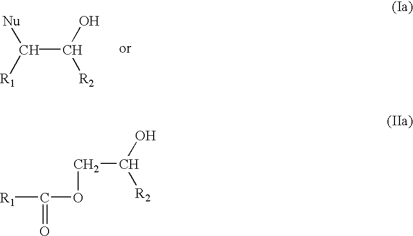

in which R₁, R₂ and Nu have the meanings given above.

Even more simply, the said compound corresponds to one of the formulae:

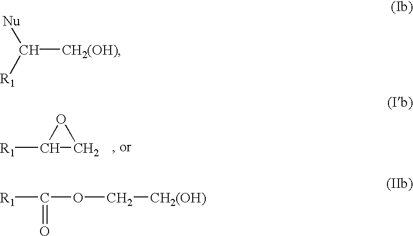

According to one embodiment variant, R₁ and R₂ or R′₁ and R′₂ together form a ring, in particular a hetero-cycle, on which the polymer is found substituted.

In particular, it is possible for (R₁ and R₂) or (R′₁ and R₂) together to form a ribose and for Nu to represent the 2′-O function protected with a protecting group such that Nu represents CH₃−C=O, for example.

In an appropriate manner, in the process for the synthesis of the nucleic acids according to the invention, the said solid support consists of a compound (I), (Ia), (Ib), (II), (IIa), (IIb) or (I′) and (I′b) according to the invention.

According to the variants most commonly used, the said nucleotide monomer reagent corresponds to the formula:

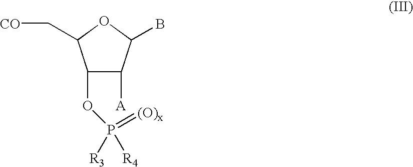

in which:

A represents H or an optionally protected hydroxyl group,

B is a purine or pyrimidine base whose exocyclic amine function is optionally protected,

C is a conventional protecting group for the 5′-OH function,

x=0 or 1, with

a) when x=1:

R₃ represents H and R₄ represents a negatively charged oxygen atom, or R₃ is an oxygen atom and R₄ represents either an oxygen atom or an oxygen atom bearing a protecting group, and

b) when x=0, R₃ is an oxygen atom bearing a protecting group and R₄ is either a halogen or a disubstituted amine group.

When x is equal to 1, R₃ is an oxygen atom and R₄ is an oxygen atom, this situation relates to the so-called phosphodiester method, when R₄ is an oxygen atom bearing a protecting group, this situation relates to the so-called phosphorotriester method.

When x is equal [lacuna] 1, R₃ is a hydrogen atom and R₄ is a hydrogen atom and R₄ is a negatively charged oxygen atom [sic], this situation relates to the so-called H-phosphosphonate method, and

when x is equal to 0, R₃ is an oxygen atom bearing a protecting group and R₄ is either [sic] a halogen, this situation relates to the so-called phosphite method and, when R₄ is a leaving group of the disubstituted amine type, this situation relates to the so-called phosphoramidite method.

The support-reagents of formula I, I′ and II according to the present invention react with the usual monomer reagents III, under the usual conditions of condensation in acidic medium in the methods for the synthesis of nucleic acids on a solid support, according to the following scheme:

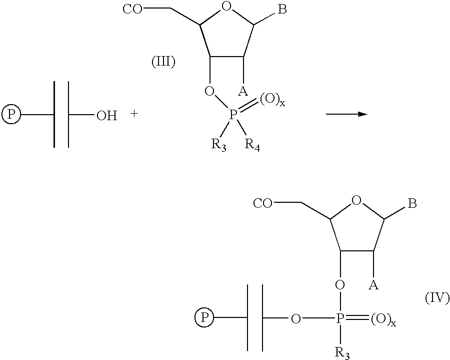

In the formulae III and IV, [symbol] , A, B, C, D, R₃, R₄ and x have the meanings given above.

In addition, under the conditions of the final detachment and deprotection step, which takes place after the last oxidation step, the oligonucleotide synthesized is separated from the support such that the (3′ or 5′) phosphate group remains attached to the support. In the case of a synthesis in the 3′→5′ direction, the reaction scheme below illustrates this last step, when the solid support of the formula I or I′ is used:

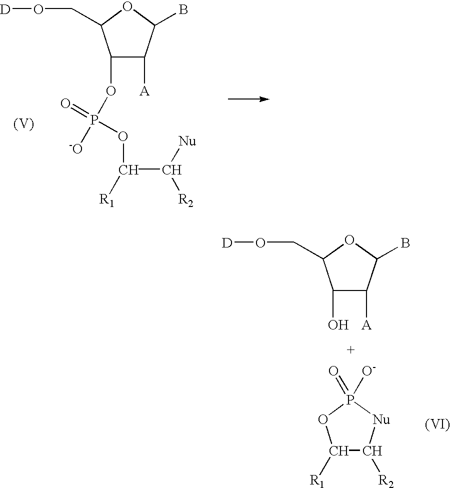

In the compounds (V) and (VI), D represents an oligonucleotide, the other parameters have the values given above.

This reaction takes place in weakly basic medium and leads to a C-5 cyclization by β-elimination.

The compounds of formula (II) in fact correspond to compounds of formula (I) in which the group Nu contains the polymer insofar as the group R₁CO-O is a nucleophilic group. When the solid support of formula II is used, this then gives the following scheme:

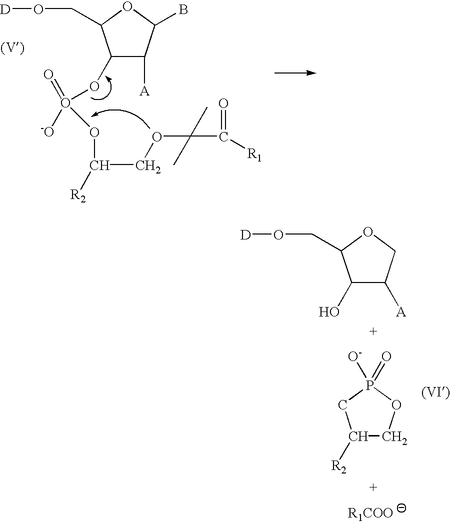

In this scheme, the polymer may be in R₂, that is to say substituted on the phosphate, ring or in R₁.

By way of polymer, mention is made of materials consisting of glass microbeads or microfibers, particularly those which are porous, silica, metal oxides or organic polymers, in particular cellulose, or optionally substituted polystyrene.

The polymer is preferably an inorganic polymer made of a glass or silica base, in particular a silica gel base.

The compounds of formulae (I), (I′) and (II) may be prepared by processes known to those skilled in the art and using available reagents.

The compounds of formula (I), (I′) or (II) may be prepared, for example, from a polymer functionalized with a COOH or NH2 group which is reacted, in a known manner, with the terminal function X=NH2 or COOH respectively of a compound

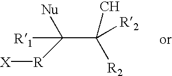

or

Groups Nu and OH are optionally protected with protecting groups;

R is a divalent residue of a hydrocarbon radical such that R₁= [symbol] −R−.

An amide bond is thus established. Obviously, in the above scheme, X−R may just as easily be substituted at R′₁.

The compounds of formulae (I′) and (II) may also be prepared according to this same type of reaction, starting with [symbol] −NH₂ and a compound where X−R is substituted to R₁, R′₁ or R′′₁ in the said formulae.

The compounds of formulae [sic] (I′) may also be prepared from

[symbol] −R₁−NH2 and from

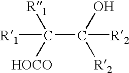

When the solid support is represented by the formula (I), it may also be prepared by a reaction of opening of the epoxide ring of formula

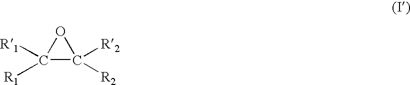

in anhydrous, acidic or basic medium, according to an SN₁ or SN₂ substitution mechanism respectively, in the presence of HNu in the medium, where Nu represents the said nucleophilic group.

When the solid support is represented by the formula (II) with [symbol] being included in R₁, it may be prepared starting with a polymer functionalized with a carboxyl function (this type of polymer is commercially available) according to the following scheme:

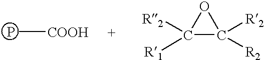

under the conditions illustrated in Example 6.

When the inorganic polymer [lacuna] made of silica, the Si—OH groups thereof may be reacted with compounds

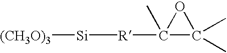

R′ is such that [symbol] —Si—R′—represents R₁ under conditions known to those skilled in the art, for example at 50° C. as illustrated in Example 1, where the compound (I) is obtained by means of the surface treatment of the solid phase with 10% glycidyloxypropyltrimethoxysilane in acetonitrile solution or by another reagent containing an epoxide, followed by an opening of the epoxide ring under controlled conditions.

The advantages of a solid support according to the invention and the use thereof in the process for the synthesis of nucleic acids, in particular the automatic synthesis, are manifold:

it is extremely simple to manufacture when compared with the usual supports;

its capacity in moles per gram is identical to that of the standard supports;

the principle thereof may be applied to all types of materials used as solid support (CPG, polymeric phases, membranes, etc.);

the parameters of the synthesis of oligonucleotides are not modified, the support is compatible with all synthesizers;

in a process for the synthesis of DNA or RNA, the deprotection step is carried out under the same conditions as for a standard support;

in a process for the synthesis of DNA or RNA, there is no additional step [lacuna] the user of the support;

the support can especially be exploited for the manufacture of oligonucleotides modified at the terminal 3′ end by using directly, in the first cycle, monomers corresponding to the desired nature of the modification;

the fact of having only one support to manufacture results in a simplification and a substantial reduction in the cost of the synthesis of the oligonucleotides;

the universal support considerably simplifies the management of the various reactors currently required for the synthesis of oligonucleotides;

lastly, the universal support makes it possible to design a multireactor synthetic system which is considerably simplified by the independence of each reactor with respect to the sequence to be synthesized.

The general formula which follows illustrates solid support compounds according to the invention:

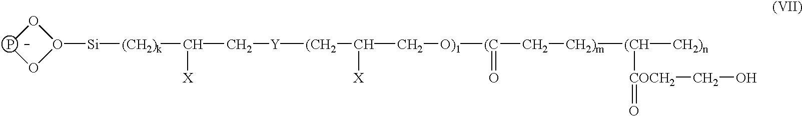

in which [symbol] —is a material consisting of glass microbeads or microfibers, silica, metal oxides, cellulose or organic polymers such as polystyrene, and in which:

k is an integer which may range from 1 to 20

l is an integer which may range from 0 to 1

m is an integer which may range from 0 to 1

n is an integer which may range from 0 to 100

X represents —H, —N(Alk)₂, —NHAcyl, —OAcyl, —SAcyl or Hal,

Y represents —H, or [sic] —O—, —NHAlk, —S— or

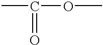

Other characteristics and advantages of the present invention will become apparent on reading the examples which follow.

In Examples 1 to 6 which follow, an APPLIED BIOSYSTEM 394® synthesizer was used. The method used is the phosphoramidite method.

The elongation is carried out in the 3′→5′ direction starting with the first nucleoside attached to the support. One synthetic cycle, corresponding to the addition of a nucleotide, also comprises three steps: unmasking, coupling and oxidation. During the unmasking step (or detritylation), the terminal 5′-hydroxyl of the oligonucleotide undergoing synthesis which is protected by the group Dmtr, is deprotected under the action of trichloroacetic acid (TCA). The trityl cation thus released has, under acidic conditions, an absorption at 498 nm, thereby making it possible to assay it and to estimate the yield for the reaction. During the condensation step, the phosphoramidite group of the monomer reagent, delivered in large excess, is activated by tetrazole and reacts with the free terminal 5′ hydroxyl to form an internucleotide bond of phosphite type.

The unstable (trivalent) phosphite is then oxidized to (pentavalent) phosphotriester in the presence of water and iodine.

The coupling yield is from 97 to 99%; it is necessary to render unreactive the 5′ hydroxyls of the unreacted oligonucleotides. This operation makes it possible to avoid extension of these truncated chains during the following cycles. This fourth step of “capping” consists of an acetylation of the 5′ hydroxyls with acetic anhydride and N-methylimidazole.

More precisely, the reagents used in the various steps are as follows:

### 1) Detritylation and coupling

Formulae A and B below schematically represent the nucleoside attached to the support and the phosphoramidite monomer reagent respectively, with

R₁=R₂=—CH(H₃)₂

R₃=—(CH₂)₂—C≡N

Scheme 1 represents the detritylation.

Scheme 2 represents the condensation.

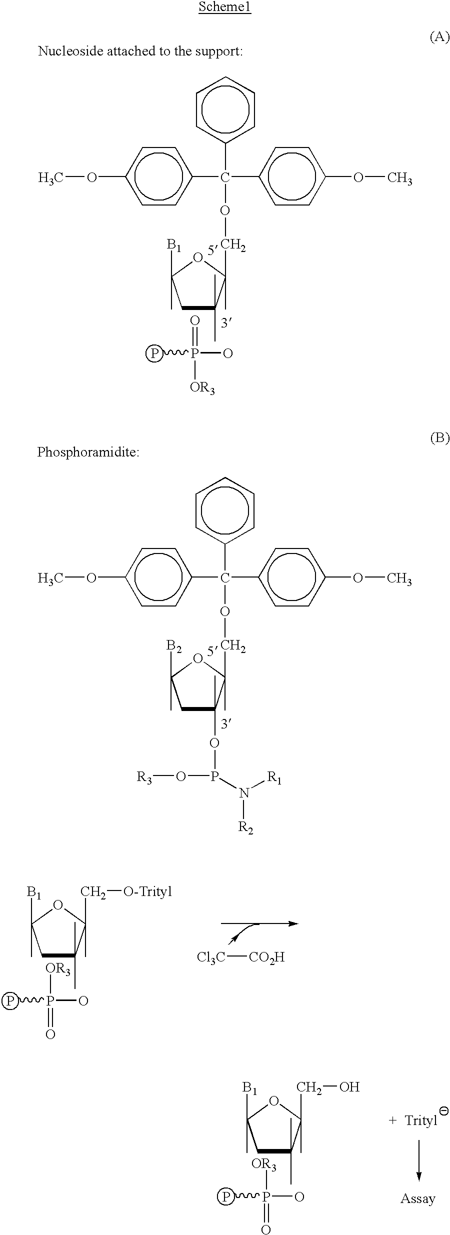

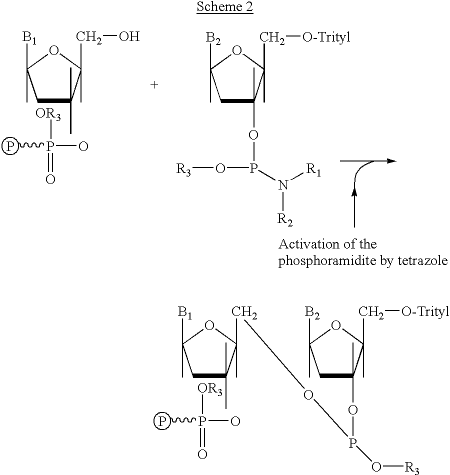

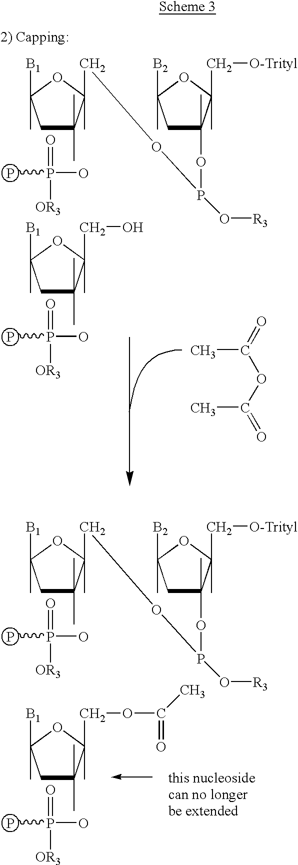

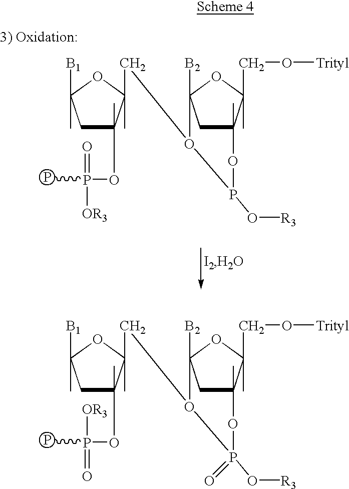

### EXAMPLE 1

1 g of porous glass powder (CPG 00350C®; f; CPG INC. USA) in 5 ml of a 10% solution of 3-glycidyloxypropyltrimethoxysilane

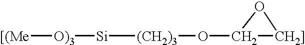

in acetonitrile, the mixture is left stand for 30 minutes at a temperature of 50° C. and the support is then separated out by filtration, washed with acetonitrile (3×5 ml) and dried under vacuum.

The number of oxy groups is determined, after opening of the epoxide ring, by means of the reaction of dimethoxytrityl chloride in pyridine followed by absorption spectrophotometric measurement of the trityl cation in a mixture of perchloric acid and ethanol at 495 nm. A capacity of 50-100 micromol per 1 g of support is obtained.

### EXAMPLE 2

The reactor is filled with 1 mg of support, obtained in Example 1, and the oligonucleotide d(ATGC) is synthesized by the standard phosphoramidite method described above, with a first step under detritylation conditions which opens the epoxide ring. After the synthesis, the oligo-CPG is heated for one hour at 100° C. in 30 microliters of concentrated aqueous ammonia solution. For the purposes of analysis, the oligonucleotide is freed, the last nucleotide of which is protected in the 5′ position, referred to hereinafter as ON-trityl for short, using HPLC on a reverse phase column. About 90% of ON-trityl oligonucleotide are obtained.

### EXAMPLE 3

The synthesis of Example 2 was performed with a synthesis of d(AGTC) by the H-phosphonate method.

As regards the synthesis of oligodeoxynucleotides by the H-phosphonate method, the following are used:

the monomers already described (formula III);

the principle of the synthesis is identical to that of the phosphoramidite method with the following few differences:

the activation agent used is either adamantoyl chloride or pivaloyl chloride,

only one oxidation step is carried out at the end of the synthesis;

the deprotection is carried out under the same conditions as for the phosphoramidites.

### EXAMPLE 4

The synthesis was performed with the same support as in Example 2, with a synthesis of AGTC in the RNA series.

As regards the synthesis of oligoribonucleotides (RNA), the monomers are of the type 5′-O-dimethoxytrityl-3′-O-β-cyanoethoxydiisopropylaminophosphine-2′-O-tert-butyldimethylsilyl-nucleosides (formula III with A=tert-butyldimethylsilyl).

The synthetic method is the so-called phosphoramidite method. As described above, the deprotection requires an additional step.

### EXAMPLE 5

The support obtained in Example 1 is washed in the reactor with an HCl solution at a concentration of 1% of dichloromethane. A support of the glycol type with Nu =Cl is obtained and the synthesis is carried out, again under the standard conditions of the phosphoroamidite method. The treatment and the detachment of the oligonucleotide is [sic] carried out as in Example 2. About 90% of ON-trityl oligonucleotide are obtained.

### EXAMPLE 6

A membrane in the form of a glass fiber disc (ø4.7 cm, 1 g, f. WATMAN)® is treated as in Example 1.

A support with a capacity of 20 μmol of oxy groups per 1 g of support is obtained.

### EXAMPLE 7

Using the disc obtained in Example 4 [sic], a disc is cut (ø4 mm, 1 mg) and the synthesis, the treatment and the detachment of the oligonucleotides d(AGTC) is [sic] performed as in Example 3.

At least 90% of ON-trityl oligonucleotide are obtained.

### EXAMPLE 8

1 g of the support, containing a carboxymethyl CPG CML® 00350C (CPG INC), is treated with 5 ml of ethylene oxide solution at a concentration of 10% of dichloromethane at a temperature of 50° C. for one hour. The support is isolated by filtration, washed with dichloromethane and dried under vacuum.

A support with a capacity of 50-100 μmol of oxy groups per 1 g of support is obtained.

1. Process for the preparation of a nucleic acid by synthesis on a solid support, characterized in that an inorganic or organic polymer is used as solid support, which polymer is connected via a divalent hydrocarbon radical to an epoxide group or a group of the glycol type, the latter group consisting of two adjacent saturated carbons on which an OH group and a nucleophilic group are respectively substituted.

2. Process according to claim 1, characterized in that the first nucleotide is advantageously attached to the solid support under the same conditions and with the same monomer reagent as for the condensation of the second nucleotide with the first nucleotide bonded to the support, which may be the conventional conditions and monomer reagent used during the synthesis of nucleic acids on a solid support, the said first nucleotide corresponding to the first nucleotide in the sequence of the said nucleic acid.

3. Process according to either of claims 1 and 2, characterized in that it comprises the following steps of:
1) condensation of the 5′ or 3′ OH group of the first nucleotide or of an oligonucleotide connected at its other 3′ or 5′ end to the said solid support, using a coupling agent, with the phosphate group optionally substituted in the 3′ or 5′ position respectively of a monomer nucleotide reagent protected in the 3′ and 5′ positions;
2) oxidation or sulfurization of the internucleotide bond of the phosphite type obtained in step 1) to a phosphate or phosphorothioate bond respectively.
3) deprotection of the 5′-O or 3′-O end of the product obtained in step 2);
4) repetition of steps 1) to 3) as many times as there are nucleotides to be added in order to synthesize the nucleic acid.

4. Process according to either of claims 1 and 2, characterized in that it comprises the following steps of:
1) condensation, using a coupling agent, of the said OH group of the said group of glycol type of the solid support with a phosphate or phosphite group optionally substituted in the 3′ or 5′ position of a monomer nucleotide reagent protected in the 5′-O and 3-O positions;
2) oxidation or sulfurization of the covalent bond of the phosphite type between the solid support and the first nucleotide obtained in step 1);
3) deprotection of the 5′-O or 3′-O end of the product obtained in step 2);
4) condensation of the 5′OH or 3′OH group of the product obtained in step 3) with the phosphate, phosphorothioate or phosphite group optionally substituted in the 3′ or 5′ position of a monomer nucleotide reagent protected in the 5′-O or 3′-O position respectively, using the said coupling agent, under the same conditions as the condensation in step 1);
5) oxidation or sulfurization of the internucleotide grouping of the phosphite phosphite [sic] type resulting from the above step into a grouping of the phosphate or phosphorothioate type respectively;
6) deprotection:ion of the 5′-O or 3′-O end of the product obtained in step 5);
7) repetition of steps (4), (5) and (6) as many times as there are nucleotides to be added in order to obtain the nucleic acid to be prepared.

5. Process according to claim 4, characterized in that it includes a final step of detachment of the nucleic acid from the support and removal of the protecting groups from the bases and, where appropriate, from the 2′-O positions of the nucleic acids.

6. Process according to either of claims 4 and 5, characterized in that it comprises a prior step of opening of the said epoxide group of the said solid support, in an anhydrous acidic medium, under the usual conditions for the deprotection of the 5′ or 3′ OH groups in order to give the said group of the glycol type of the solid support.

7. Compounds represented by the following formulae: 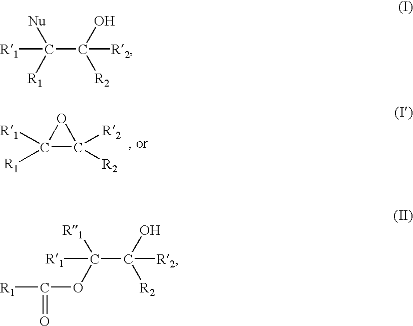
in which:
one of R₁, R′₁, R′′₁, R₂ and R′₂ represents an inorganic or organic polymer or a hydrocarbon radical substituted with an inorganic or organic polymer, and the others are identical or different and represent, independently of each other, H or an inert group such as an alkyl group which is optionally substituted, in particular with one or more halogen(s),
Nu represents a nucleophilic group such as NH_{2,} Halogen —OAlk, —SAlk, —NHAlk, —NHAc, —OAc, —SAc or —N(Alk)₂, where Alk and Ac respectively represent an alkyl and acyl group, which is optionally substituted, in particular with one or more halogen(s).

8. Compounds according to claim 7, characterized in that Nu represents —N(Alk)₂, —NHAc, —OAc, —SAc or a halogen, where Alk and Ac respectively represent a C₁ to C₄ alkyl and acyl group optionally substituted with one or more halogen(s).

9. Compounds according to claim 7 or 8, characterized in that the said solid support corresponds to one 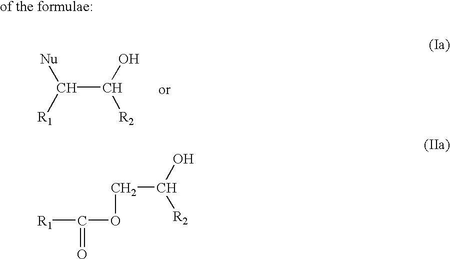
in which R₁, R₂ and Nu have the meanings given in claim 7.

10. Compound according to claim 9, characterized in that the said compound corresponds to one of the formulae:

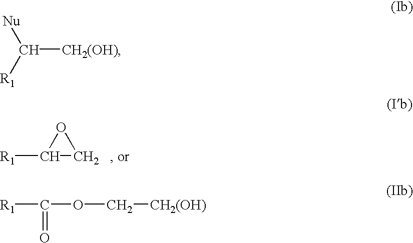

11. Compound according to one of claims 7 to 9, characterized in that (R₁ and R₂) or (R′₁ and R′₂) together form a ring, in particular a heterocycle, on which the polymer is found substituted.

12. Composition according to claim 11, characterized in that (R₁ and R₂) or (R′₁ and R₂) together form a ribose ring and Nu represents the 2′-O function protected with a protecting group such as

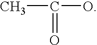

13. Process according to one of claims 1 to 6, characterized in that the said solid support consists of a compound according to one of claims 7 to 10. a compound according to one of claims 7 to 10.

14. Process according to one of claims 2 to 6 and 13, characterized in that the said nucleotide monomer reagent corresponds to the formula: 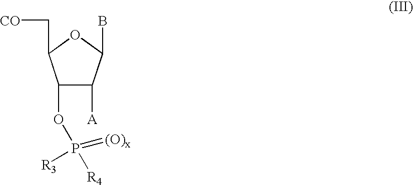
in which:
A represents H or an optionally protected hydroxyl group,
B is a purine or pyrimidine base whose exocyclic amine function is optionally protected,
C is a conventional protecting group for the 5′-OH function,
x=0 or 1, with
a) when x=1:
R₃ represents H and R₄ represents a negatively charged oxygen atom, or R₃ is an oxygen atom and R₄ represents either an oxygen atom or an oxygen atom bearing a protecting group, and
b)when x=0, R₃ is an oxygen atom bearing a protecting group and R₄ is either a halogen or a disubstituted amine group.

15. Process according to claim 14, characterized in that it is a phosphoramidite synthesis process in which the monomer reagent corresponds to the formula (III) with x=0, R₃ is an oxygen atom bearing a protecting group and R₄ is a disubstituted amine group.

16. Process according to one of claims 1 to 6 and 13 to 15, characterized in that the polymer is in the form of glass microbeads or microfibers, in particular porous ones, silica, metal oxides, cellulose or organic polymer, in particular cellulose.

17. Process according to one of claims 1 to 6 and 13 to 16, characterized in that the polymer is an inorganic polymer made, in particular, of a glass or silica base.
